(19) United States
(12) Patent Application Publication
(10) Pub. No.: US 2016/0232317 A1
(43) Pub. Date: Aug. 11, 2016

(54) APPARATUS FOR AND METHOD OF PROVIDING BIOLOGICAL INFORMATION

(71) Applicant: SAMSUNG ELECTRONICS CO., LTD., Suwon-si (KR)

(72) Inventors: Younho KIM, Hwaseong-si (KR); Yongjoo KWON, Yongin-si (KR); Sunkwon KIM, Suwon-si (KR); Sangyun PARK, Hwaseong-si (KR)

(73) Assignee: SAMSUNG ELECTRONICS CO., LTD., Suwon-si (KR)

(21) Appl. No.: 14/847,411

(22) Filed: Sep. 8, 2015

(30) Foreign Application Priority Data
Feb. 5, 2015 (KR) 10-2015-0018136

(51) Int. Cl. G06F 19/00; A61B 5/024; A61B 5/00

(52) U.S. Cl. CPC G06F 19/3418; A61B 5/0077; A61B 5/024; A61B 5/4884; A61B 5/0022; A61B 2560/0252; A61B 2562/0204

(57) ABSTRACT

A biological information providing apparatus. The apparatus includes a requester configured to request, from a server, a user's environment information, the environment information having been collected when a fluctuation in biological information of the user occurred, a receiver configured to receive the environment information from the server, an editor configured to edit the environment information, and a content provider configured to provide biological information content based on the edited environment information.

### RELATED APPLICATION

This application claims the benefit of Korean Patent Application No. 10-2015-0018136, filed on Feb. 5, 2015, in the Korean Intellectual Property Office, the disclosure of which is incorporated herein in its entirety by reference.

### BACKGROUND

1. Field

The present disclosure relates to apparatuses for and methods of generating and providing biological information content based on environment information of a user when a fluctuation in user's biological information occurs.

2. Description of the Related Art

Along with the advancements in medical science and the increased interest in healthcare, research on healthcare and health management techniques have become important. In particular, as factors such as a blood pressure, blood sugar, or stress from a user's biological information are important with regard to various diseases, methods for measuring such factors have been developed.

Recently, analyzing the causes of changes of factors such as stress, blood pressure, or blood sugar as well as measuring these factors have become important. However, existing health management systems only provide measurements of such factors, without providing information about the causes of disease occurrence.

### SUMMARY

Provided are apparatuses for and methods of generating and providing biological information content based on environment information of a user collected in a vicinity around a user when a fluctuation in user's biological information occurs.

Additional aspects will be set forth in part in the description which follows and, in part, will be apparent from the description, or may be learned by practice of the presented exemplary embodiments.

According to an aspect of an exemplary embodiment, a biological information providing apparatus can include a requester configured to request, from a server, a user's environment information, the environment information having been collected when a fluctuation in biological information of the user occurred; a receiver configured to receive the environment information from the server; an editor configured to edit the environment information; and a content provider configured to provide biological information content based on the edited environment information.

The environment information can include information about at least one of a photographed image, a video, a sound, a temperature, an illuminance, a smell, and weather collected in a vicinity around the user when the fluctuation in biological information occurred.

The editor is configured to receive selection information about an item to be included in the biological information content, and edit the environment information according to the selection information about the item.

The editor is configured to acquire playback range selection information from the photographed image and adjust a photographed image playback time according to the playback range selection information.

The content provider is configured to receive text information and generate the biological information content by inserting the text into the environment information.

The requester is configured to display fluctuation identification information relating to the fluctuation in biological information, receives selection information relating to the fluctuation identification information, and requests information according to the selection information from the server.

The requester is further configured to display a level of the fluctuation in biological information in a fluctuation identification information display region, and the level is based on at least one of a blood pressure, a blood sugar level, and a stress index of the user.

The content provider is configured to transmit the biological information content to at least one of an SNS server and a medical institution server.

According to another aspect of an exemplary embodiment, a biological information providing method can include requesting, from a server, a user's environment information, the environment information having been collected when a fluctuation in biological information of the user occurred; receiving the environment information from the server; editing the environment information; and providing biological information content based on the edited environment information.

The environment information can include information about at least one of a photographed image, a video, a sound, a temperature, an illuminance, a smell, and weather collected in a vicinity around the user.

The editing of the environment information can include receiving selection information about an item to be included in the biological information content; and editing the environment information according to the selection information about the item.

The editing of the environment information can further include receiving playback range selection information from the photographed image; and adjusting a photographed image playback time according to the playback range selection information.

The providing of the biological information content can include receiving text information; and generating the biological information content by inserting the text information into the environment information.

The requesting can include displaying fluctuation identification information related to the fluctuation in biological information; receiving selection information with respect to the fluctuation identification information; and requesting, by a requester, information according to the selection information from the server.

The providing of the biological information content can include transmitting the biological information content to at least one of an SNS server and a medical institution server.

According to yet another aspect of an exemplary embodiment, a biological information providing system can include a biological information measuring apparatus configured to measure biological signal of a user, determine whether a fluctuation in biological information of the user occurred based on a variation of the biological signal, and collect environment information of the user when it is determined that the fluctuation in the biological information occurred; a server configured to receive and store the environment information from the biological information measuring apparatus; and a biological information providing apparatus configured to request the environment information from the server, receive the environment information from the server, and provide biological information content by editing the environment information.

The biological information measuring apparatus measures a heart rate of the user, calculates a stress index of the user from the heart rate, and determines whether the fluctuation in the biological information occurred based on the calculated stress index.

The biological information measuring apparatus determines a level of the fluctuation in biological information based on the calculated stress index, and sets a time for collecting the environment information according to the determined level.

The environment information can include at least one of information about a photographed image, a video, a sound, a temperature, an illuminance, a smell, and weather collected in a vicinity around the user, and the biological information providing apparatus changes a playback range of the photographed image.

According to a still further aspect of an exemplary embodiment, a biological information measuring method can include measuring a biological signal of a user; determining whether a fluctuation in biological information of the user occurred based on a variation of the biological signal; and collecting environment information of the user when it is determined that the fluctuation in the biological information occurred.

The method can further include transmitting the environment information to a server.

The environment information can include at least one of information about a photographed image, a video, a sound, a temperature, an illuminance, a smell, and weather collected in a vicinity around the user

The method can further include measuring a heart rate of the user, calculating a stress index of the user from the heart rate, and determining whether the fluctuation in the biological information occurred based on the calculated stress index.

The method can further include determining a level of the fluctuation in biological information based on the calculated stress index, and collecting the environment information for an amount of time, wherein the amount of time is selected according to the determined level.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a view of a biological information providing system according to an exemplary embodiment;

FIG. 2 is a view illustrating a biological information providing process executed in the biological information providing system according to an exemplary embodiment;

FIG. 3 is a flow chart of a biological information collecting method executed by a biological information collecting device according to an exemplary embodiment;

FIGS. 4 and 5 are graphs illustrating examples of calculating a stress index from a heart rate of a user;

FIG. 6 is an exemplary table showing options for collecting environment information via a biological information collecting device;

FIG. 7 is a conceptual block diagram of a biological information providing apparatus according to an exemplary embodiment;

FIG. 8 is a graph illustrating an example of displaying identification information about a fluctuation of the biological information;

FIG. 9 is a graph illustrating another example of displaying identification information about a fluctuation of the biological information;

FIG. 10 is a view illustrating another example of displaying identification information about a fluctuation of the biological information;

FIG. 11 is a view illustrating another example of displaying identification information about a fluctuation of the biological information;

FIG. 12 is a view illustrating an example of editing environment information via an editor having an editing function;

FIG. 13 is an exemplary view illustrating biological information content generated by a content provider;

FIG. 14 is another exemplary view illustrating biological information content generated by a content provider;

FIG. 15 is another exemplary view illustrating biological information content generated by a content provider; and

FIG. 16 is a flow chart of a biological information providing method executed by a biological information providing apparatus according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of” when preceding a list of elements, modify the entire list of elements and do not modify the individual elements of the list.

In the following exemplary embodiments, terms such as “first”, “second”, “A”, “B”, “(a)”, “(b)”, and so forth are used only for distinguishing one component from another component, rather than for restrictive meanings.

It will be understood that when a layer, region, or component is referred to as being “connected to” or “bonded to” another layer, region, or component, it can be directly or indirectly “connected to” or “bonded to” the other layer, region, or component. That is, for example, intervening layers, regions, or components may be present.

An expression used in the singular encompasses the expression of the plural, unless it has a clearly different meaning in the context. Also, throughout the specification, when a portion “includes” an element, another element may be further included, rather than excluding the existence of the other element, unless otherwise described.

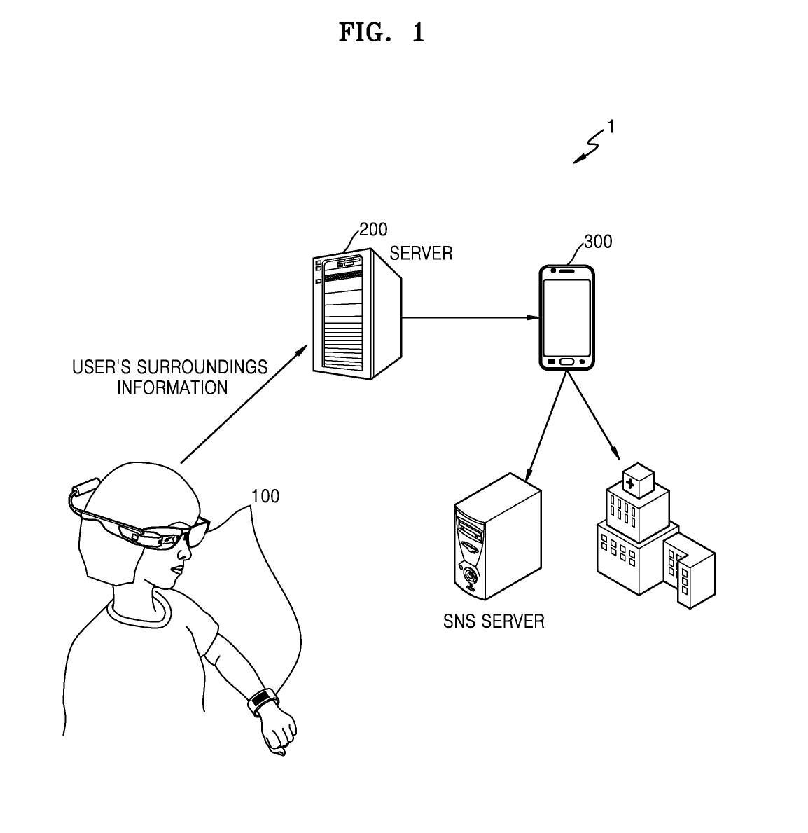

FIG. 1 is a view of a biological information providing system 1 according to an exemplary embodiment.

Referring to FIG. 1, the biological information providing system 1 may include a biological information collecting device 100, a server 200, and a biological information providing apparatus 300. Although the biological information collecting device 100 and the biological information providing apparatus 300 are shown as being separated from each other in FIG. 1, the exemplary embodiment is not limited thereto. Thus, the biological information collecting device 100 and the biological information providing apparatus 300 may be implemented in the same hardware unit. In this case, an application to perform functions of the biological information collecting device 100 and the biological information providing apparatus 300 may be stored in the hardware unit. As another example, the biological information collecting device 100 and the biological information providing apparatus 300 may be implemented into hardware units separated from each other. In this case, an application to perform a function of the biological information collecting device 100 and an application to perform a function of the biological information providing apparatus 300 may be respectively stored in each of the hardware units. As another example, the server 200 and the biological information collecting device 100 may be implemented as one hardware unit. In this case, the server 200 may be used as a memory or a database for storing collected biological information, information on whether a fluctuation of the biological information occurred or not, environment information of a user, and so on. As another example, the server 200 and the biological information providing apparatus 300 may be implemented as one hardware unit.

For convenience of explanation, only the hardware components shown in FIG. 1 will be described. However, other general purpose hardware components, for example, an AP (access point) and a network hub, may be included in the biological information providing system 1 of FIG. 1. Moreover, the biological information collecting device 100 and the biological information providing apparatus 300 may be connected to the server 200 through a network, thereby enabling server-client communication.

The network between the server 200 and the biological information collecting device 100 and the biological information providing apparatus 300 may be a closed network such as a local area network (LAN), a wide area network (WAN), or an open network such as the Internet. The Internet may be used for various services according to the TCP/IP protocol and the upper-layer protocol and may be a world-wide open computer network structure providing a hypertext transfer protocol (HTTP), a telnet protocol, a file transfer protocol (FTP), a domain name system (DNS) service, a simple mail transfer protocol (SMTP), an simple network management protocol (SNMP), a network file service (NFS), a network information service (NIS), etc. Furthermore, if the biological information collecting device 100 and the biological information providing apparatus 300 are a mobile communication terminal or a mobile terminal such as a smart phone and a wearable device, the network may be a wireless network such as a mobile communication network or a Wi-Fi network.

The biological information collecting device 100 may measure a biological signal of a user and may determine whether or not a fluctuation in biological information of the user occurred based on a variation of the biological signal. In addition, when it is determined that a fluctuation occurred in the biological information of the user, environment information of the user may be collected and transmitted to the server 200. The biological information collecting device 100 may be a mobile device or a wearable device such as a smart watch or a pair of glasses. An exemplary operation method of the biological information collecting device 100 will be described below.

The server 200 may store environment information received from the biological information collecting device 100. In addition, if the server 200 receives a request from the biological information providing apparatus 300, the server 200 may send information to the biological information providing apparatus 300 in response to the request.

The server 200 may be connected to a number of clients (including the biological information collecting device 100 and the biological information providing apparatus 300) that are not able to identify and/or other server through a network generally such as the Internet. The server 200 may be a computer system configured to accept an operation request of a client or another web server, and determine and provide a corresponding operation result. Computer software (a server program) may be installed on the server 200. Furthermore, the server 200 may store a series of application programs and various databases (hereinafter, “DB”). The DB may be embodied in the server or outside the server 200. The DB may store and manage information of a user using the biological information providing system 1, information about a fluctuation of the biological information of each user, and environment information of each user.

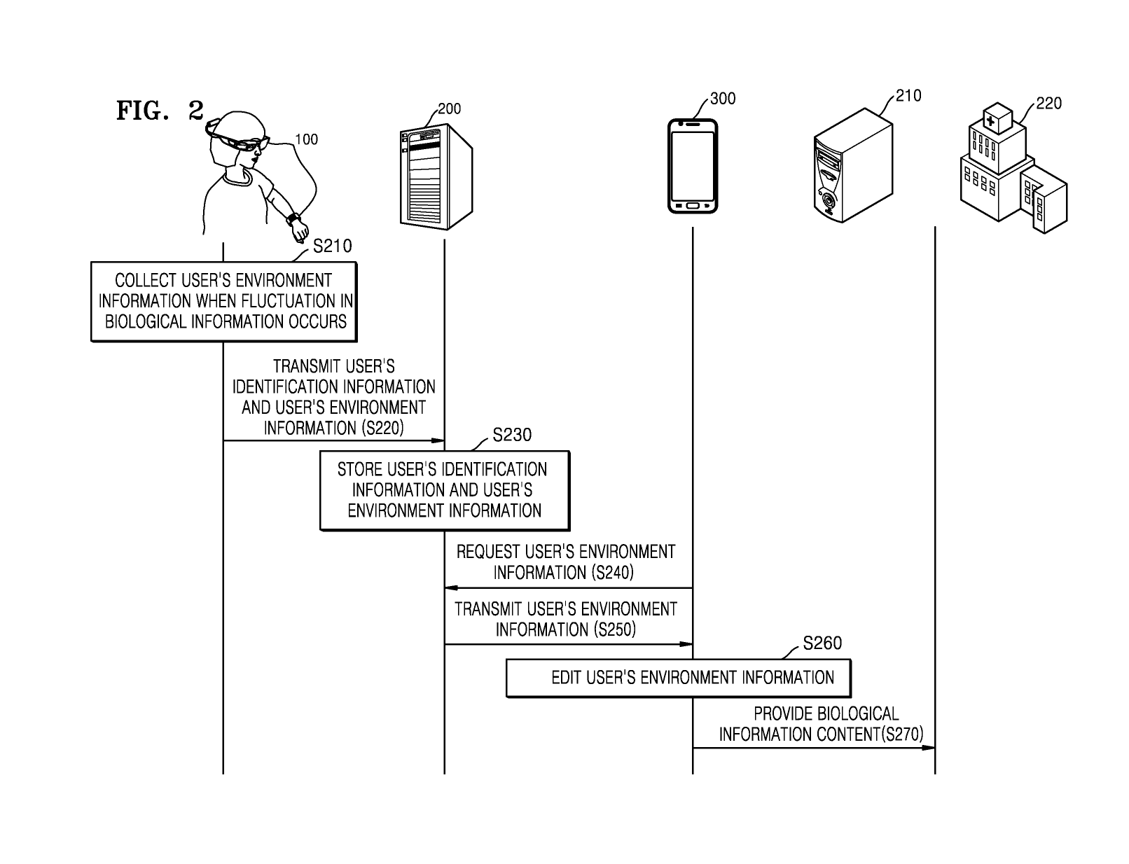

FIG. 2 is a view illustrating a biological information providing process executed in the biological information providing system 1 according to an exemplary embodiment.

Referring to FIG. 2, in operation S210, the biological information collecting device 100 may collect environment information of the user when a fluctuation in biological information of the user occurred. The biological information may reflect a health state of the user, and may include at least one of a blood pressure, a blood sugar level, and a stress index. However, this is just an example and the biological information is not limited thereto. Furthermore, the fluctuation of the biological information may be a fluctuation in the blood pressure, the blood sugar level or the stress index with respect to a critical range, or a rate of change of the blood pressure, the blood sugar level, or the stress index. The fluctuation of the biological information may be detected by the biological information collecting device 100 and will be described in detail below.

The environment information of the user may include at least one of information about a photographed image, a sound, a temperature, an illuminance, a smell, and weather collected in a vicinity around the user. In order to collect the environment information, the biological information collecting device 100 may include a camera, a recorder, a temperature sensor, an illuminance sensor, a smell sensor, and so on.

In operation S220, the biological information collecting device 100 may transmit user identification information and environment information to the server 200.

The user identification information is specific information to distinguish the users and may include for example, a user name or ID. Furthermore, the user identification information may include a user password in addition to the user name or the ID. The user identification information may be obtained from login information or user's input, but is not limited thereto and may also be obtained by various methods.

The biological information collecting device 100 may transmit the user identification information and the environment information to the server 200. At this time, the biological information collecting device 100 may also transmit to the server 200 fluctuation identification information about the fluctuation of the biological information corresponding to the environment information. The fluctuation identification information may include time information when the fluctuation occurred. Furthermore, the fluctuation identification information may include information about a level of the fluctuation based on at least one of the blood pressure, the blood sugar level, and the stress index.

In operation S230, the server 200 may store the user identification information and the environment information received from the biological signal measuring apparatus 100. The server 100 may map and store the user identification information and the environment information. Furthermore, if the biological information collecting device 100 transmits the fluctuation identification information to the server 100, the server 100 may also map the fluctuation identification information with the environment information and store the result.

In operation S240, the biological information providing apparatus 300 may request from the server 200 the environment information. In operation S240, the biological information providing apparatus 300 may receive the fluctuation identification information such as the time when the fluctuation occurred and the level of the fluctuation. Furthermore, in response to a selection input of the user, the biological information providing apparatus 300 may request information corresponding to the selection from the server 200.

In operation S250, in response to the request issued by the biological information providing apparatus 300, the server 200 may transmit the environment information to the biological information providing apparatus 300. Furthermore, in operation S250, the server 200 may transmit the fluctuation identification information such as the time when the fluctuation occurred and the level of the fluctuation to the biological information providing apparatus 300.

In operation S260, the biological information providing apparatus 300 may edit the environment information received from the server 200. In operation S270, the biological information providing apparatus 300 may provide biological information content. As illustrated in FIG. 2, in operation S270, the biological information providing apparatus 300 may provide the biological information content to a social network service (SNS) server 210 or a medical institution server 220. The SNS server 210 may be a server providing various social network services such as FACEBOOK, TWITTER, PINTEREST, KAKAO TALK, blog services, or the like. Furthermore, the medical institution server may be a server operated by the medical institution recording and managing medical information about patients such as a clinic or a hospital. FIG. 2 illustrates a case in which the biological information providing apparatus 300 transmits the biological information content to another device in operation S270, but the exemplary embodiment is not limited thereto. As another example, in operation S270, the biological information providing apparatus 300 may store the biological information content in the biological information providing apparatus 300 without transmitting to another apparatus.

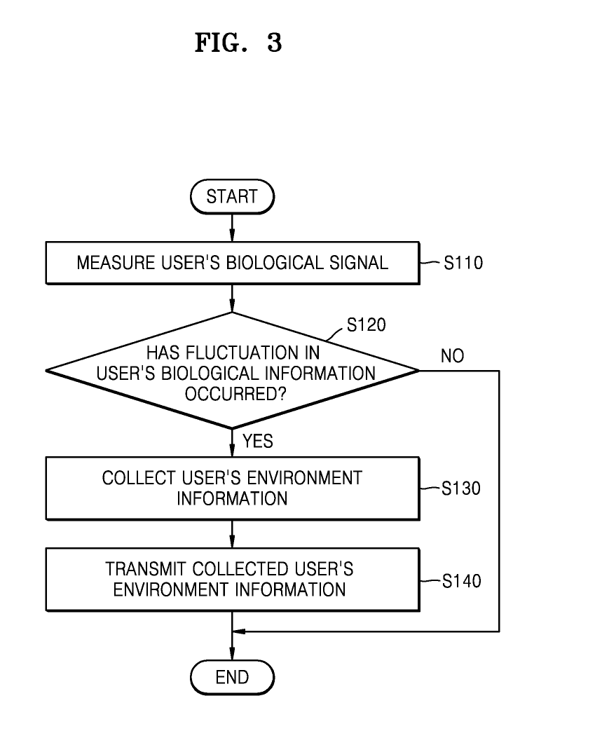

FIG. 3 is a flow chart of a biological information collecting method executed by the biological information collecting device 100 according to an exemplary embodiment.

Referring to FIG. 3, the biological information collecting method executed by the biological information collecting device 100 may include operation S110 of measuring biological signal of the user, operation S120 of determining whether a fluctuation of the biological information of the user occurred based on a variation of the biological signal, operation S130 of collecting environment information of the user when the fluctuation of the biological information occurred, and operation S140 of transmitting the collected environment information to the server 200.

In operation S110 of measuring the biological signal, the biological signal may be a heart rate, a galvanic skin resonance, a skin spectrum, and so on.

In operation S120, it is determined whether the fluctuation of the biological information occurred from the measured biological signal. Therefore, operation S120 may include calculating the biological information from the measured biological signal. The biological information may include information capable of reflecting a health state of the user such as a blood pressure, a blood sugar level, or a stress index.

The biological information collecting device 100 may calculate the biological information from the biological signal by various methods. For example, the biological information collecting device 100 may calculate the stress index from the user's heart rate. The biological information collecting device 100 may detect a degree of activation of a sympathetic nerve from the heart rate. Furthermore, the biological information collecting device 100 may derive the stress index from the degree of activation of the sympathetic nerve.

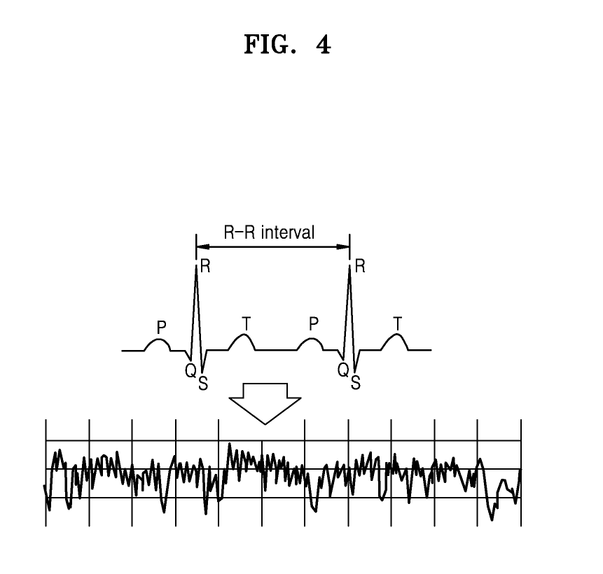

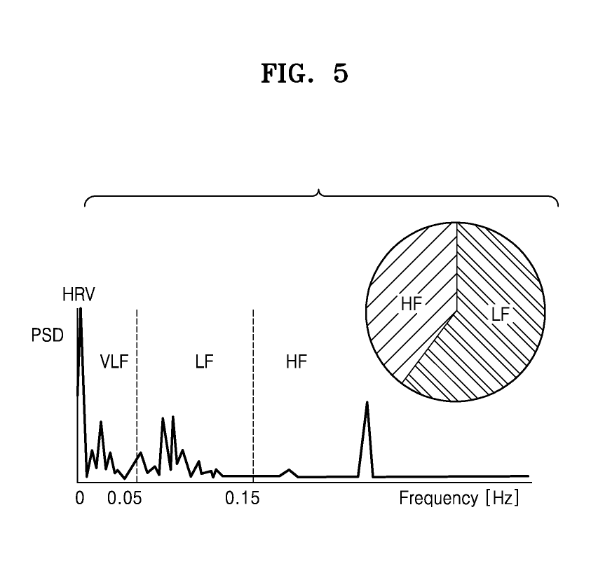

FIGS. 4 and 5 are graphs illustrating examples of calculating the stress index from the heart rate of the user.

Referring to FIG. 4, the biological information collecting device 100 may collect an R-R interval (RRI) variation from an electrocardiogram (ECG) signal of the user. A graph of FIG. 4 represents RRI variation data during a predetermined period. FIG. 4 may represent a graph of an ECG wave, where R is a point corresponding to the peak of a QRS complex of the ECG wave; and the R-R interval is the interval between successive Rs, as is known in the art. The RRI variation may change according to an activity of the autonomic nervous system of the user. The autonomic nervous system may include a sympathetic nerve and a parasympathetic nerve.

Referring to FIG. 5, the biological information collecting device 100 may analyze a frequency spectrum from the RRI variation data. The RRI variation may be slower with respect to the sympathetic nerve and may be faster with respect to the parasympathetic nerve. When the sympathetic nerve is stimulated, a reaction time is delayed about five seconds compared to a case when the parasympathetic nerve is stimulated. Thus, according to analyzing results of a frequency spectrum, a low-frequency band (LF) may be related to the user's sympathetic nerve and a high-frequency band (HF) may be related to the user's parasympathetic nerve. LF and HF may be divided in intervals of about 0.1 Hz. The sympathetic nerve is activated when the user is excited or nervous, and the parasympathetic nerve is activated when the user is calm.

The biological information collecting device 100, according to the analyzing results of the frequency spectrum with respect to the RRI variation data, may calculate the stress index from a ratio of an integrated value of the LF and HF. In detail, each of the integrated values of the LF and HF represents degree of activation of the sympathetic nerve. In other words, the biological information collecting device 100 may calculate the stress index according to the degree of activation of the sympathetic nerve. When the user's stress is in a normal range, the ratio of the integrated value of the LF and that of the HF is about 6:4. When the user's stress increases, the ratio of the integrated value of the LF and that of the HF may increase.

FIGS. 4 and 5 illustrate examples of calculating the stress index from the heart rate of the user, but the exemplary embodiment is not limited thereto. For example, the biological information collecting device 100 can irradiate light on the skin and analyze a spectrum, and thus may obtain the distribution amount of skin oil or hyaluronic acid on the skin. In addition, the biological information collecting device 100 may calculate the stress index from the distribution amount of the skin oil or the hyaluronic acid. As another example, the biological information collecting device 100 may be attached to the user's arm and detect a pulse wave of the user and thus may calculate the user's blood pressure. As another example, the biological information collecting device 100 may irradiate light on the user's skin and measure the amount of reflected light. Furthermore, the biological information collecting device 100 may calculate the stress index of the user or blood sugar level by using Raman spectroscopy.

When a variation occurs in the biological information such as the blood pressure, blood sugar level, or stress index, the biological information collecting device 100 may determine that a fluctuation of the biological information occurred. Furthermore, the biological information collecting device 100 may collect environment information after determining that the fluctuation occurred (S130). The environment information may include a photographed image, a sound, a temperature, an illuminance, a smell, weather, and so on collected in the vicinity around the user, but is not limited thereto. The biological information collecting device 100 may include a camera, a temperature sensor, a recorder, an illuminance sensor, and so on to obtain the environment information.

The biological information collecting device 100 may set an option for collecting the environment information according to a kind of the fluctuation of the biological information. For example, when a level of the fluctuation is provided, the biological information collecting device 100 may set an option for collecting the environment information according to the fluctuation level.

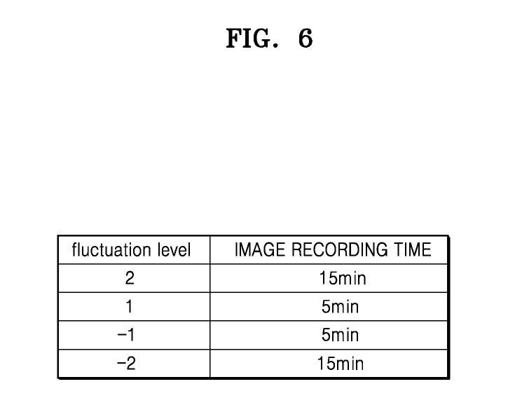

FIG. 6 is an exemplary table showing options for collecting environment information via the biological information collecting device 100.

Referring to FIG. 6, the biological information collecting device 100 may set a time for photographing an image of environment information according to the fluctuation level. In other words, the biological information collecting device 100 may set the time for photographing the image to be relatively longer when the blood pressure, the blood sugar level or the stress index of the user is greatly different from those in a normal range. Thus, the biological information collecting device 100 may collect a relatively large amount of environment information with respect to a fluctuation having a greater influence on the user's health or emotion.

In operation S140 of FIG. 3, the biological information collecting device 100 may transmit the collected environment information to the server 200. At this time, the biological information collecting device 100 may further transmit user identification information to the server 200. Furthermore, the biological information collecting device 100 may transmit fluctuation identification information to the server 200. The fluctuation identification information may include information such as a time when the fluctuation occurred and a level of the fluctuation.

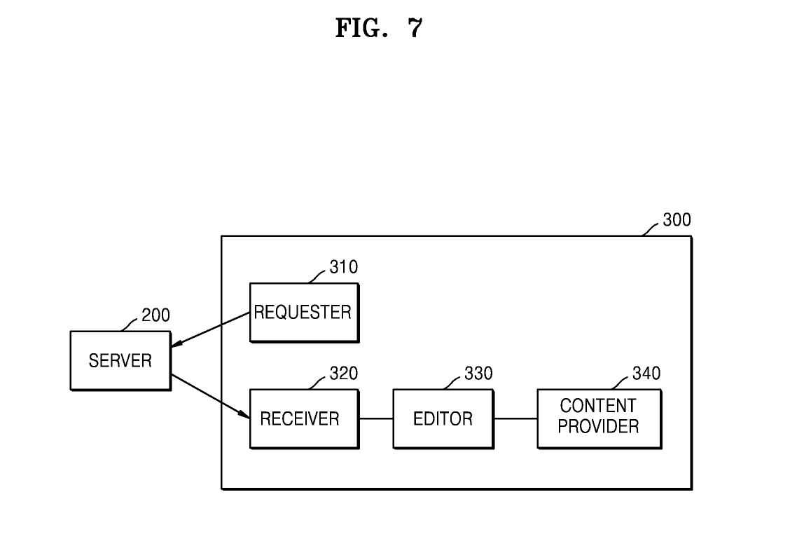

FIG. 7 is a conceptual block diagram of a biological information providing apparatus 300 according to an exemplary embodiment. The biological information providing apparatus 300 of FIG. 7 may be a smart phone, a tablet PC, a PC, a smart TV, a mobile phone, a personal digital assistant (PDA), a laptop computer, a media player, a micro server, a global positioning system (GPS) device, a electronic book terminal, a digital broadcasting terminal, a navigation, a kiosk, an MP3 player, a digital camera, and other mobile or non-mobile computing device, but is not limited thereto.

Referring to FIG. 7, the biological information providing apparatus 300 according to an exemplary embodiment may include a requester 310 to request information about a fluctuation of biological information of a user, a receiver 320 to receive from the server 200 environment information collected in a vicinity around the user when the fluctuation of the biological information occurred, an editor 330 to edit the environment information, and a content provider 340 to provide biological information content based on the edited environment information.

The requester 310 may provide the user with a fluctuation list of fluctuations of the biological information corresponding to the environment information. The requester 310 may receive a desired selection of the fluctuation list from the user. The requester 310 is a structure that provides a user with information and receives a selection from the user and may include a monitor, a keyboard, a touch screen, and so on.

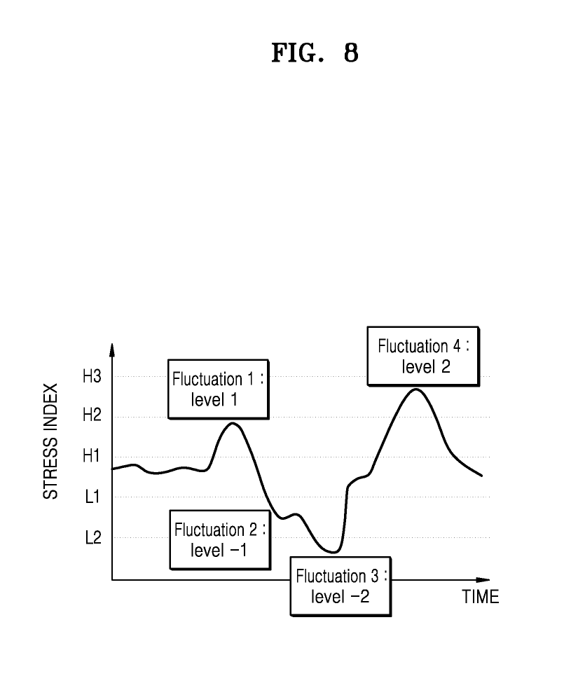

FIG. 8 is a graph illustrating an example of displaying fluctuation identification information.

Referring to FIG. 8, the requester 310 may display a graph showing a variation of a stress index of the user over time. Furthermore, the requester 310, in regions in which the stress index changes in the graph, may display the fluctuation identification information corresponding to the stress index variation. Fluctuation levels may be displayed in respective identification information display regions. The fluctuation level may be provided based on a stress index at a time when the fluctuation occurred as described above. For example, as illustrated in FIG. 8, level 1 is provided to fluctuation 1 that occurred when the stress index is between H1 and H2. Furthermore, level 2 is provided to fluctuation 2 that occurred when the stress index is between H2 and H3. Level-1 is provided to fluctuation 2 that occurred when the stress index is between L1 and L2. In addition, Level-2 is provided to fluctuation 3 that occurred when the stress index is less than L2. FIG. 8 illustrates only an exemplary embodiment, and the number of fluctuation levels and a providing method thereof may vary. Furthermore, FIG. 8 illustrates a stress index as an example but the graph is not limited thereto. For example, the blood pressure or blood sugar level may be used instead of the stress index, and accordingly, the fluctuation level is also provided according to the biological information instead of the stress index.

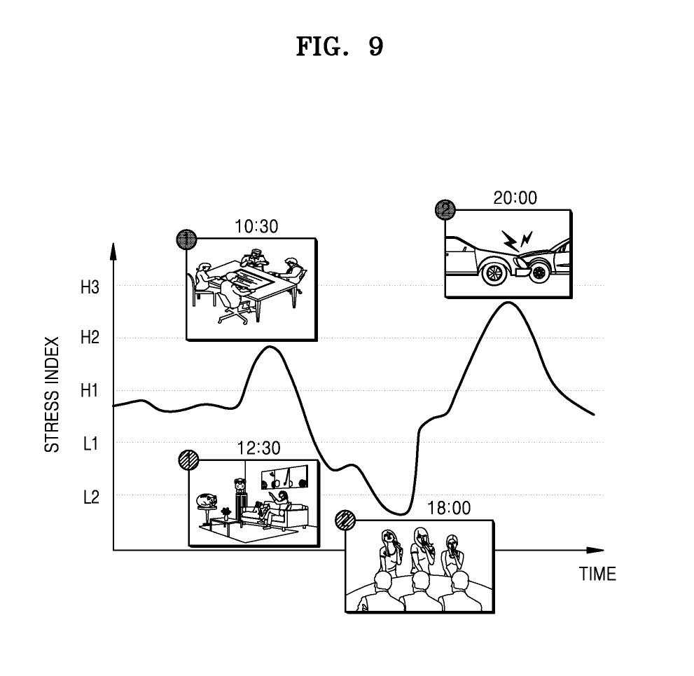

FIG. 9 is a graph illustrating another example of displaying fluctuation identification information.

Referring to FIG. 9, the requester 310 may display a graph showing a variation of a stress index of the user over time. Furthermore, the requester 310, in regions in which the stress index changes in the graph, may display the fluctuation identification information corresponding to the stress index variation. When a fluctuation in biological information occurred, a part of collected environment information may be displayed in the identification information display region. For example, if the environment information includes a photographed image taken around the user, one scene of the photographed image may be captured and displayed in the identification information display region, but the exemplary embodiment is not limited thereto. For example, as a part of the environment information, a sound value in decibels (dB), an illuminance value, a temperature value, weather information, and so on collected when the fluctuation of the biological information occurred may be displayed in the identification information display region. Furthermore, a time or the point in time when the fluctuation occurred may be displayed in the identification information display region. A level of the fluctuation may be displayed in the identification information display region. The level may be provided based on the stress index when the fluctuation occurred as described above. FIG. 9 illustrates a stress index as an example but the exemplary embodiment is not limited thereto. For example, the blood pressure or blood sugar level may be used instead of the stress index, and also the level is provided based on the new biological information value.

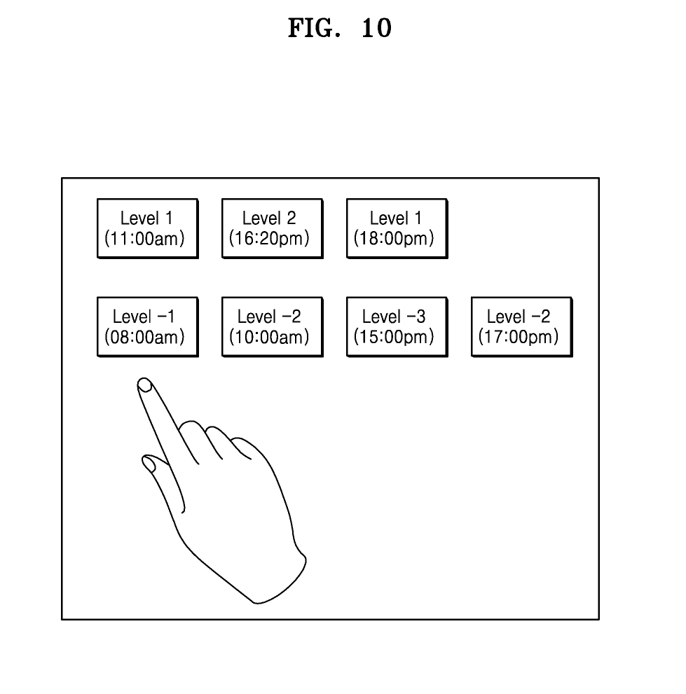

FIG. 10 is a view illustrating another example of displaying fluctuation identification information about a fluctuation of the biological information.

Referring to FIG. 10, the requester 310 may request displaying identification information corresponding to each fluctuation of the biological information. Fluctuation levels provided according to the stress index and so on may be displayed in a identification information display region. The fluctuation level may be provided based on other biological information value such as the blood pressure or the blood sugar level. Furthermore, a time or the point in time when the fluctuation occurred may be displayed in the identification information display region.

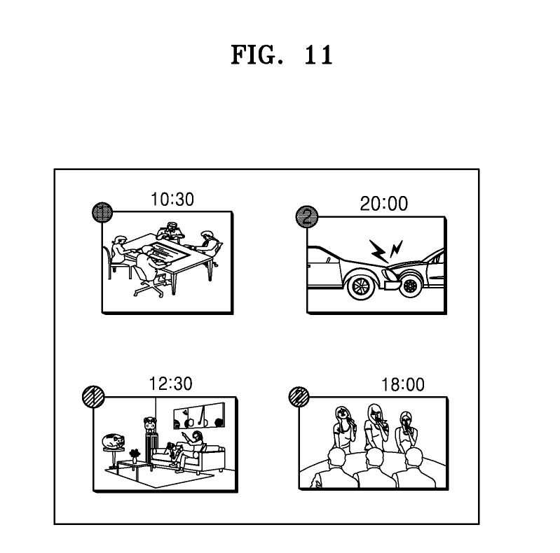

FIG. 11 is a view illustrating another example of displaying fluctuation identification information about a fluctuation of the biological information.

Referring to FIG. 11, the requester 310 may request displaying identification information corresponding to each fluctuation of the biological information. Fluctuation levels provided according to the stress index and so on may be displayed in identification information display regions. The fluctuation level may be provided based on other biological information value such as the blood pressure or the blood sugar level. Furthermore, a time or the point in time when the fluctuation occurred may be displayed in the identification information display regions. When a fluctuation of the biological information occurred, a part of collected environment information may be displayed in each of the identification information display regions. For example, if the environment information includes a photographed image taken around the user, one scene of the photographed image may be captured and displayed in the identification information display region, but the exemplary embodiment is not limited thereto. For example, as a part of the environment information, a sound value in decibels (dB), an illuminance value, a temperature value, weather information, and so on collected when the fluctuation occurred may be displayed in the identification information display region.

Examples in which the requester 310 provides a selection list of the fluctuations to a user are illustrated in FIGS. 8 to 11. However, this is just an example and methods of providing the fluctuation identification information by the requester 310 may vary. The requester 310 may provide user with the selection list of the fluctuations and receive a user's selection. In addition, the requester 310 may request information corresponding to the user's selection from the server 200. Therefore, the requester 310 may transmit a request signal to the server 200, and range information about the requested information may be included in the request signal.

Referring again to FIG. 7, when the server 200 receives the request signal from the requester 310, the server 200 may transmit information corresponding to the request signal to the receiver 320. The receiver 320 may provide the editor 330 with the environment information received from the server 200. The requester 310 and the receiver 320 may communicate with the server 200 by various methods. For example, when the biological information providing apparatus 300 is a non-mobile computer device such as a desktop computer or a smart TV, the biological information providing apparatus 300 may communicate with the server 200 by wire communication. As another example, when the biological information providing apparatus 300 is a mobile computer device such as a smart phone, a tablet PC, a smart watch, or Google Glasses, the biological information providing apparatus 300 may communicate with the server 200 by using a wireless communication.

The requester 310 and the receiver 320 may include WI-FI chips, BLUETOOTH chips, wireless communication chips, or NFC chips in order to communicate with the server 200. The WI-FI chip and the BLUETOOTH chip may perform communication via a WI-FI method and a BLUETOOTH method, respectively. Furthermore, the wireless communication chip may include a chip performing the communication according to various communication standards such as IEEE, ZIGBEE, 3RD GENERATION (3G), 3RD GENERATION PARTNERSHIP PROJECT (3GPP), or LONG TERM EVOLUTION (LTE). The requester 310 and the receiver 320 may use different hardware resources or may share an identical hardware resource in order to communicate with the server 200.

The editor 330 may edit the environment information provided from the receiver 320. For example, if the environment information includes a plurality of information items such as a photographed image, a sound, a temperature, an illuminance, a smell, and weather, the editor 330 may receive selection information of the items to be included in the biological information content from the user. Furthermore, the editor 330 may select and edit environment information according to the selection information.

The editor 330 may provide the user with the environment information and an editing operation may be performed by the user via the editor 330. The editor 330 is a structure for providing information to the user and receiving a user's input and may include a monitor, a keyboard, a touch screen, and so on. The editor 330 may share the constitution of the monitor, the keyboard, the touch screen, and so on with the requester 310. However, the editor 330 and the requester 310 may use different hardware resources.

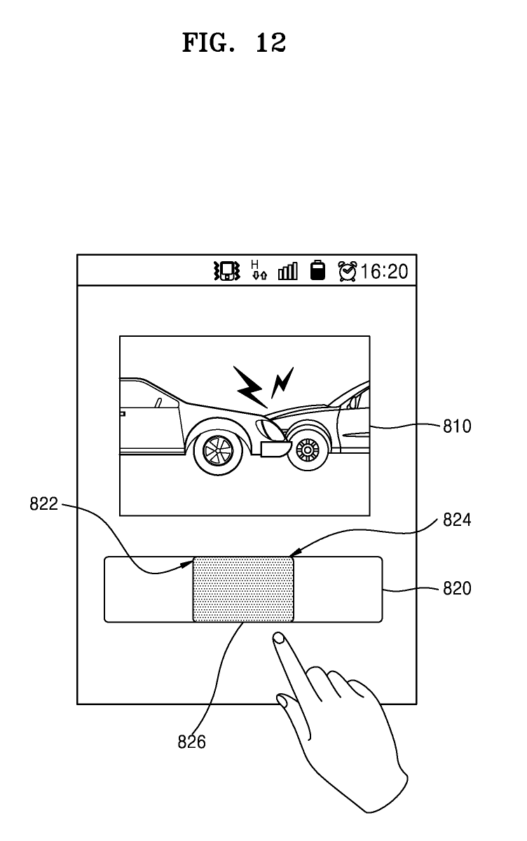

FIG. 12 is a view illustrating an example of editing environment information via the editor 330 having an editing function.

Referring to FIG. 12, the editor 330 may display a photographed image or video acquired from the environment information. A playback section of the photographed image or video may be displayed via a bar type icon 820. The editor 330 may receive a user's input selecting a start point 822 and an end point 824 of an edited image or video in the bar type icon 820, and may edit the playback section of the photographed image or video corresponding to the user's input. FIG. 12 is just an example and editing environments provided by the editor 330 may vary. For example, the editor 330 may support an image capturing function to capture one scene from the photographed image, or a still image from a video. Furthermore, the editor 330 may support an editing function to edit a playback range of sounds recorded around the user. In addition, the editor 330 may support an editing function for editing a screen on which information such as a temperature, an illuminance, a smell, or weather is displayed.

The content provider 340 may receive edited environment information from the editor 330, and may generate biological information content based on the edited environment information. The biological information content may represent content generated by using the environment information collected when a fluctuation in the biological information occurs. The content provider 340 may generate the biological information content in various forms such as a diary, and social network posts by using the edited environment information.

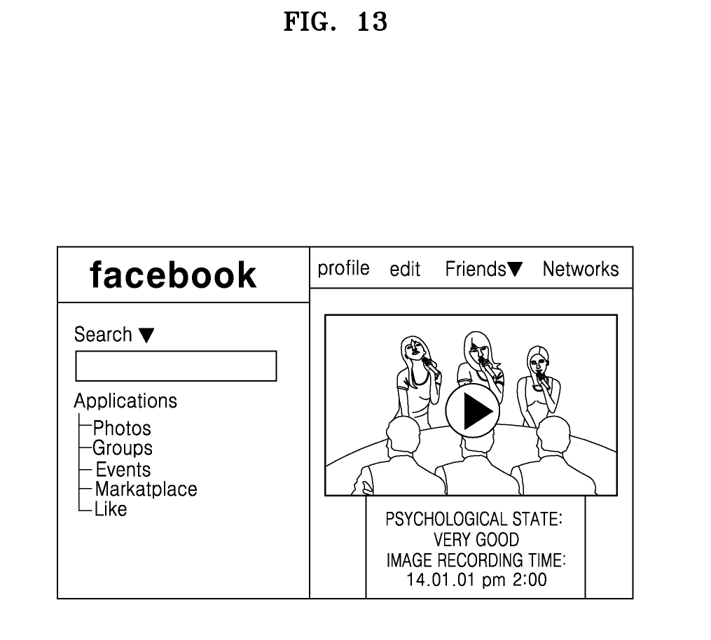

FIG. 13 is an exemplary view illustrating biological information content generated by a content provider 340.

Referring to FIG. 13, an image edited by the editor 330 may be provided as the biological information content. The content provider 340 may provide the edited image itself as the biological information content. However, other information may also displayed with the edited image. For example, when the edited image is collected as illustrated in FIG. 13, information about the user's health state may be displayed together with the edited image. The information about the user's health state may be derived based on a level of the fluctuation. Furthermore, time information about when the edited image is taken may be simultaneously displayed.

In another exemplary embodiment, the editor 330 may provide an edited photo as the biological information content. Also, information about the user's emotional state may be simultaneously displayed with the edited photo.

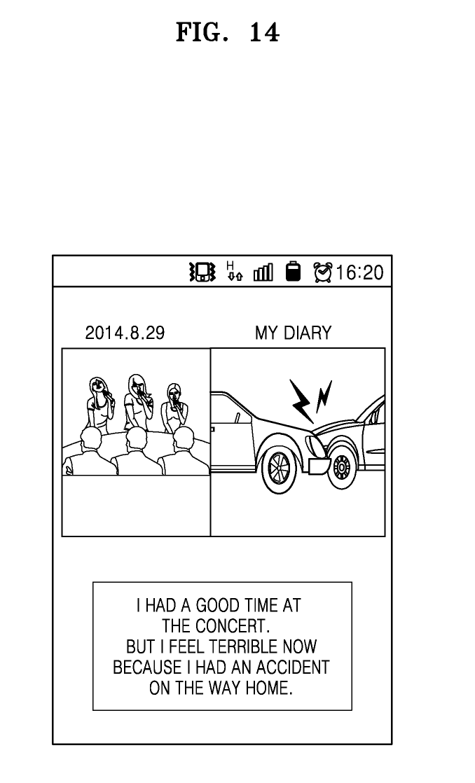

FIG. 14 is another exemplary view illustrating biological information content generated by a content provider 340.

Referring to FIG. 14, the content provider 340 may generate diary-type biological information content by using a photo, an edited image or a captured image. Therefore, the content provider 340 may receive a user's text input and may generate the diary-type biological information content by combining the received text input information with the edited image or the captured image. FIG. 14 illustrates two captured images but the number of captured images is not limited thereto. Furthermore, text may be combined with information about the edited image other than the captured image, sound, smell, weather, sound, or an illuminance. When the content provider 340 combines sound and text, an icon capable of reproducing sound with text may be displayed together. As another example, the content provider 340 may display text and may simultaneously reproduce edited sound.

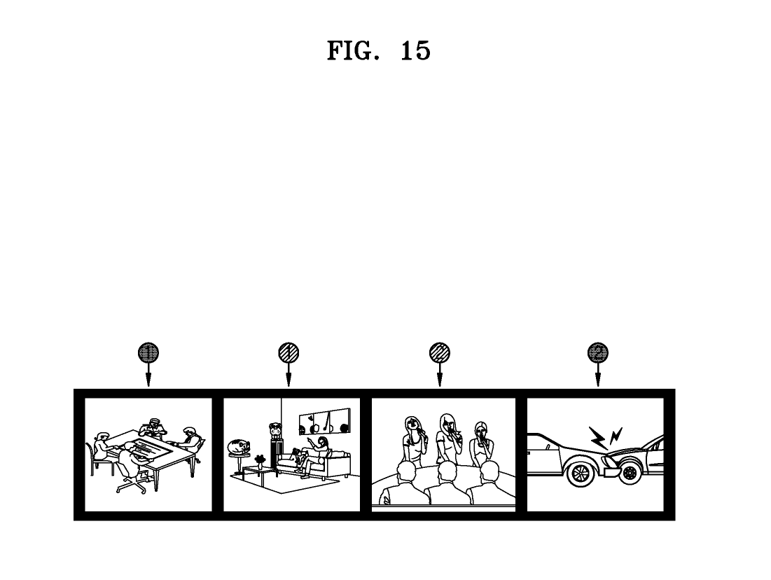

FIG. 15 is another exemplary view illustrating biological information content generated by a content provider 340.

Referring to FIG. 15, the content provider 340 may generate a moving image by combining a plurality of edited photographed images. Therefore, the content provider 340 may provide a user with an environment in which the plurality of edited photographed images are combined. The content provider 340 may receive option information with respect to an arrangement order of the plurality of photographed images and may combine the plurality of photographed images according to the option information. Therefore, the user may generate a moving image in which events that affected his/her health state are recorded. Furthermore, the content provider 340 may transmit the generated moving image to an SNS server and may post the moving image on the Internet. In addition, the content provider 340 may provide the moving image to a medical institution server as reference for treatment.

The biological information content generated by the content provider 340 may be stored in the biological information providing apparatus 300. The user may personally analyze a stress cause based on the stored biological information content. Furthermore, the user may have a chance to check again the environment information that is collected when he or she feels good.

The content provider 340 may provide an SNS server 210 or a medical institution server 220 with the generated biological information content. The SNS server 210 may be a server providing various social network services such as FACEBOOK, TWITTER, PINTEREST, KAKAO TALK, and blog services, or the like. Furthermore, the medical institution server may be a server that is managed by the medical institution such as a clinic or a hospital where patients' medical information is recorded and stored. The medical institution may easily determine a fluctuation factor of biological information such as user's stress, blood pressure, blood sugar through the biological information content provided by the content provider 340. The medical institution may use the information for medical treatment and care programs for patients. Furthermore, the content provider 340 transmits the biological information content to the SNS server, and thus, the user may share the environment information regarding factors such as the user's stress level, the blood pressure, and blood sugar with other people. The user may variously express his/her personality and feelings while sharing events that had affected his/her health state.

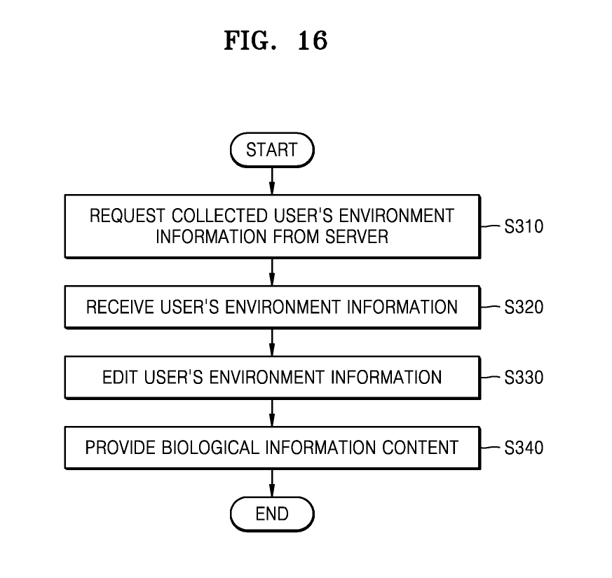

FIG. 16 is a flow chart of a biological information providing method executed by the biological information providing apparatus 300 according to an exemplary embodiment.

Referring to FIG. 16, the biological information providing method of the biological information providing apparatus 300 may include operation S310 of requesting collected environment information from the server 200 when the fluctuation in the biological information of the user occurred, operation S320 of receiving the environment information from the server 200, operation S330 of editing the environment information, and operation S340 of providing biological information content based on the edited environment information.

In operation S310, the requester 310 may provide the user with a list of fluctuations of the biological information corresponding to the environment information. A fluctuation of the biological information list may be provided as illustrated in FIGS. 8 to 11. In operation S310, the requester 310 may receive a desired selection of the fluctuation of the biological information from the user. The requester 310 may generate a request signal requesting information corresponding to the user's selection and may transmit the signal to the server 200. In operation S320, the receiver 320 may receive the environment information transmitted by the server 200. Along with the environment information, fluctuation identification information such as a fluctuation level or a time when the fluctuation occurred may be included in the information received by the receiver 320.

In operation S330, the editor 330 may edit the environment information. The editor 330 may provide an interface for changing a playback range of the photographed image as illustrated in FIG. 12, but the exemplary embodiment is not limited thereto. The interface provided by the editor 330 may be of different types. Furthermore, the editor 330 may provide an interface for changing other environment information such as the sound, temperature, weather, illuminance, or smell.

In operation S340, the content provider 340 may provide biological information content based on the edited environment information. The biological information content may be generated by various methods as illustrated in FIGS. 13 to 15. The biological information content may be a post for simply posting the edited environment information. As another example, the biological information content may be a content such as a diary in which text information is combined with the edited environment information. Furthermore, the biological information content may be an image content in which a plurality of photographed images are combined.

As described above, the one or more of the above exemplary embodiments can be implemented through computer readable code/instructions in/on a medium, e.g., a computer readable medium, to control at least one processing element to implement any above described exemplary embodiment. The medium can correspond to any medium/media permitting the storage and/or transmission of the computer readable code.

The computer readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as the Internet transmission media. Thus, the medium may be such a defined and measurable structure including or carrying a signal or information, such as a device carrying a bitstream according to one or more exemplary embodiments. The media may also be a distributed network, so that the computer readable code is stored/transferred and executed in a distributed fashion. Furthermore, the processing element could include a processor or a computer processor, and processing elements may be distributed and/or included in a single device.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that this description is intended to be illustrative, and not to limit the scope of the claims.

What is claimed is:

1. A biological information providing apparatus, the apparatus comprising:
a requester configured to request, from a server, a user's environment information, the environment information having been collected when a fluctuation in biological information of the user occurred;
a receiver configured to receive the environment information from the server;
an editor configured to edit the environment information; and
a content provider configured to provide biological information content based on the edited environment information.

2. The apparatus of claim 1, wherein the environment information comprises information about at least one of a photographed image, a video, a sound, a temperature, an illuminance, a smell, and weather collected in a vicinity around the user when the fluctuation in biological information occurred.

3. The apparatus of claim 2, wherein the editor is configured to receive selection information about an item to be included in the biological information content, and edit the environment information according to the selection information about the item.

4. The apparatus of claim 2, wherein the editor is configured to acquire playback range selection information from the photographed image and adjust a photographed image playback time according to the playback range selection information.

5. The apparatus of claim 1, wherein the content provider is configured to receive text information and generate the biological information content by inserting the text into the environment information.

6. The apparatus of claim 1, wherein the requester is configured to display fluctuation identification information relating to the fluctuation in biological information, receive selection information relating to the fluctuation identification information, and request information according to the selection information from the server.

7. The apparatus of claim 6, wherein the requester is further configured to display a level of the fluctuation in biological information in a fluctuation identification information display region, and wherein the level is based on at least one of a blood pressure, a blood sugar level, and a stress index of the user.

8. The apparatus of claim 1, wherein the content provider is configured to transmit the biological information content to at least one of an SNS server and a medical institution server.

9. A biological information providing method, the method comprising:
requesting, from a server, a user's environment information, the environment information having been collected when a fluctuation in biological information of the user occurred;
receiving the environment information from the server;
editing the environment information; and
providing biological information content based on the edited environment information.

10. The method of claim 9, wherein the environment information comprises information about at least one of a photographed image, a video, a sound, a temperature, an illuminance, a smell, and weather collected in a vicinity around the user.

11. The method of claim 10, wherein the editing of the environment information comprises receiving selection information about an item to be included in the biological information content; and editing the environment information according to the selection information about the item.

12. The method of claim 10, wherein the editing of the environment information further comprises receiving playback range selection information from the photographed image; and adjusting a photographed image playback time according to the playback range selection information.

13. The method of claim 9, wherein the providing of the biological information content comprises receiving text information; and generating the biological information content by inserting the text information into the environment information.

14. The method of claim 9, wherein the requesting comprises displaying fluctuation identification information related to the fluctuation in biological information; receiving selection information with respect to the fluctuation identification information; and requesting, by a requester, information according to the selection information from the server.

15. The method of claim 9, wherein the providing of the biological information content comprises transmitting the biological information content to at least one of an SNS server and a medical institution server.

16. A biological information providing system, the system comprising:
a biological information measuring apparatus configured to measure biological signal of a user, determine whether a fluctuation in biological information of the user occurred based on a variation of the biological signal, and collect environment information of the user when it is determined that the fluctuation in the biological information occurred;
a server configured to receive and store the environment information from the biological information measuring apparatus; and
a biological information providing apparatus configured to request the environment information from the server, receive the environment information from the server, and provide biological information content by editing the environment information.

17. The system of claim 16, wherein the biological information measuring apparatus is configured to measure a heart rate of the user, calculate a stress index of the user from the heart rate, and determine whether the fluctuation in the biological information occurred based on the calculated stress index.

18. The system of claim 17, wherein the biological information measuring apparatus is configured to determine a level of the fluctuation in biological information based on the calculated stress index, and set a time for collecting the environment information according to the determined level.

19. The system of claim 16, wherein the environment information comprises at least one of information about a photographed image, a video, a sound, a temperature, an illuminance, a smell, and weather collected in a vicinity around the user, and the biological information providing apparatus changes a playback range of the photographed image.

20. A biological information measuring method, comprising:
measuring a biological signal of a user;
determining whether a fluctuation in biological information of the user occurred based on a variation of the biological signal; and
collecting environment information of the user when it is determined that the fluctuation in the biological information occurred.

21. The method of claim 20, further comprising:
transmitting the environment information to a server.

22. The method of claim 20, wherein the environment information comprises at least one of information about a photographed image, a video, a sound, a temperature, an illuminance, a smell, and weather collected in a vicinity around the user

23. The method of claim 20, further comprising:
measuring a heart rate of the user,
calculating a stress index of the user from the heart rate, and determining whether the fluctuation in the biological information occurred based on the calculated stress index.

24. The method of claim 20, further comprising:
determining a level of the fluctuation in biological information based on the calculated stress index, and collecting the environment information for an amount of time, wherein the amount of time is selected according to the determined level.
